(19) United States
(12) Patent Application Publication
(10) Pub. No.: US 2017/0151009 A1
(43) Pub. Date: Jun. 1, 2017

(54) Device For Cutting Off And Removing Calcified Tissue From A Heart Valve

(71) Applicants: Bernard Pain, Monistrol sur Loire (FR); Marco Vola, Saint Priest en Jarez (FR)

(72) Inventors: Bernard Pain, Monistrol sur Loire (FR); Marco Vola, Saint Priest en Jarez (FR); Enrico Pasquino, Marentino (IT)

(21) Appl. No.: 15/316,350

(22) PCT Filed: Jun. 4, 2015
(86) PCT No.: PCT/FR2015/051490

§ 371 Date: Dec. 6, 2016

(30) Foreign Application Priority Data
Jun. 5, 2014 (FR) 1455145

(51) Int. Cl. A61B 18/08; A61B 17/22; A61B 17/00

(52) U.S. Cl. CPC A61B 18/082; A61B 17/00234; A61B 17/22; A61B 2017/00243; A61B 2018/00369

(57) ABSTRACT

A device for cutting off and removing calcified tissue from a heart valve, including a tubular body suitable for being inserted by way of a minimally invasive or endoscopic channel in relation to a previously inserted guide wire suitable for passing through the flaps of the valve above the portion where the calcified tissue is to be removed. One of the ends of the body, viewed at the flaps, has a profiled tip made of flexible material and arrangements for the lateral projection of a cutting member for cutting the tissue. The end of the body has, upstream from the arrangements for the lateral projection of the cutting member, a laterally offset rounded area defining, on the same side as the arrangements, a recessed portion and, on the opposite side, a projecting portion suitable for bearing upon the flaps during the cutting procedure under the effect of a rotational movement of the body that can be actuated using only one hand by way of a handle located externally in order to decalcify the whole valve. The arrangements are in communication with a mechanism for sucking away the cut tissue.

### FIELD OF THE INVENTION

The invention relates to the art of endoscopic surgery, and more specifically to a device for cutting off and removing calcified tissue from heart valves.

### BACKGROUND OF THE INVENTION

In the art of cardiac surgery, it is known, that during open heart aortic valve replacement surgery, i.e. with extracorporeal circulation, the first step is to decalcify and remove those portions of the diseased valve that are likely to generate paravavular leaks at the level of the prosthesis to be implanted. It is therefore necessary to cut out this diseased and calcified tissue. This is usually performed by means of surgical scissors, with the requirement of having to retain those pieces that have been cut out, in order to avoid losing them within the blood system. Such a surgical procedure is relatively simple by means of open surgery in that there is ample access to the rib cage to be cut.

This type of surgery is being performed more and more by means of minimally invasive surgery with incisions into the chest wall (mini-sternotomies or mini-thoracotomies), or possibly exclusively thoracoscopically. Such a surgical procedure requires the taking of more precautions given the risk that pieces of cut tissue may be lost within the patient's blood system.

### SUMMARY OF THE INVENTION

From this state of the art, one of the problems that the invention proposes to solve, in the case of minimally invasive surgery, is to be able to cut out calcified native valve tissue, with one gesture, without the extraction and iterative reintroduction of instruments into the patient's thorax, and with only one hand, thereby avoiding any risk of said cut tissue being left within the blood system.

To solve this problem, a device for cutting off and removing calcified tissue from a heart valve was designed and developed, which device is remarkable in that it comprises a tubular body suitable for being inserted by means of a minimally invasive or endoscopic channel in relation to a previously inserted guide wire and suitable for passing through the flaps of the valve above the portion where the calcified tissue is to be removed, one of the ends of said body viewed at said flaps, having a profiled tip made of soft material and arrangements for the lateral projection of a cutting member for cutting said tissue, that can be actuated using only one hand by means of a handle located externally, said arrangements being in communication with means for sucking away and removing the cut tissue.

The result of these features is that the device is not obstructive at the level of the ascending aorta such that the decalcification procedure can be performed by means of open surgery or minimally invasive surgery and with the positioning in parallel of an optical device for use in video-assistance or thoracoscopy.

To solve the problem posed of being able to cut away calcified tissue from a diseased valve in a single gesture and using only one hand, the end of the body has, upstream from the arrangements for the lateral projection of the cutting member, a laterally offset rounded area defining, on the same side as the arrangements, a recessed portion and, on the opposite side, a projecting portion suitable for bearing upon the flaps during the cutting procedure under the effect of a rotational movement of the body in order to decalcify the whole valve. The body is mounted free to rotate relative to the handle such that it is capable of being driven manually by means of a wheel.

To solve the problem posed of allowing a surgeon to see the area to be treated and to gradually verify the results obtained from the intervention the device has a vision system opening into the recessed portion of the rounded area, at the level of the cutting member. Advantageously, the body has a means of maintaining the vision system in a stable position during the cutting procedure.

To resolve the problem posed of being able to remove defective tissue, the arrangements for the lateral projection of the cutting member consist of an opening formed at the end of the body, at the tip.

The cutting member conforms to the cutting of tissue by translation and/or rotation against the edge of the opening, from the center to the periphery of the valve, as a result of lateral pressure.

In one embodiment, the cutting member has successive active areas with different features, in order to achieve, first a coarse cut, then a fine cut.

According to another feature, the device is combined with a washing/suction system that is suitable for removing tissue and calcification debris upon the ablation thereof, said system being put into operation when the cutting member is actuated, and obstructing the opening in order to create a seal with respect to the exterior thereof.

Advantageously, the soft tip is made using a different color to that of the decalcifying area and has a graduation for the precise positioning of the working area with the cutting member.

### BRIEF DESCRIPTION OF THE DRAWINGS

The Invention is Described Below in More Detail Using the Figures of the Drawings Attached, Wherein:

FIG. 1 is a perspective view prior to the assembly of the main components of the device according to the invention,

FIG. 2 is a perspective view of the device after the assembly of its components, the device being represented in two separate parts before assembly,

FIG. 3 is a perspective view showing the device, the vision system and the guide wire,

FIG. 4 is a cross-sectional view (A-A in FIG. 2) in schematic form and at a very large scale of the device,

FIGS. 5 to 11 show the main steps for the ablation of calcified tissue at the level of the flaps of a native aortic valve by means of the device according to the invention,

FIG. 12 is a perspective view with a partial cut of the device according to an alternative embodiment,

FIG. 13 shows the use of the device according to the embodiment illustrated in FIG. 12.

### DETAILED DESCRIPTION OF THE INVENTION

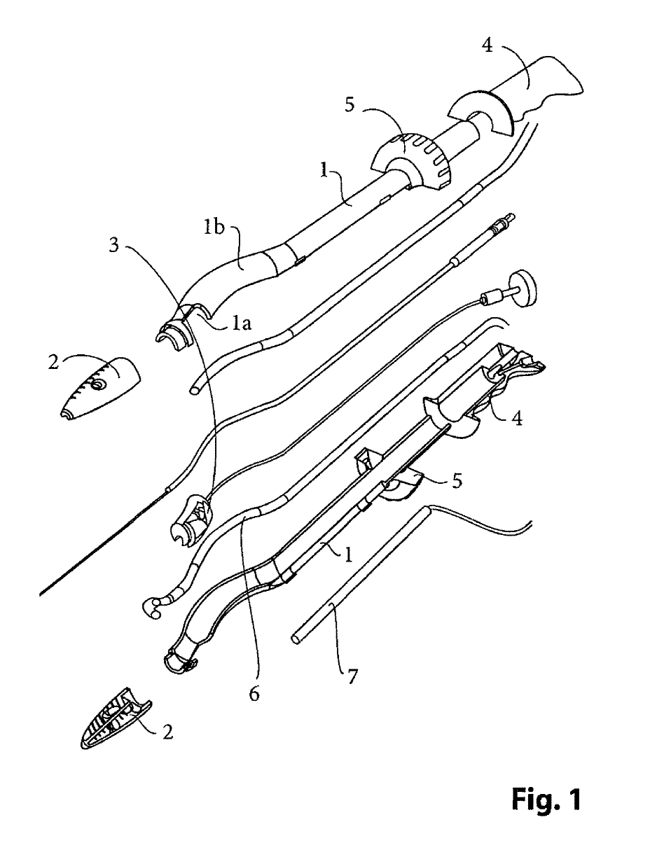

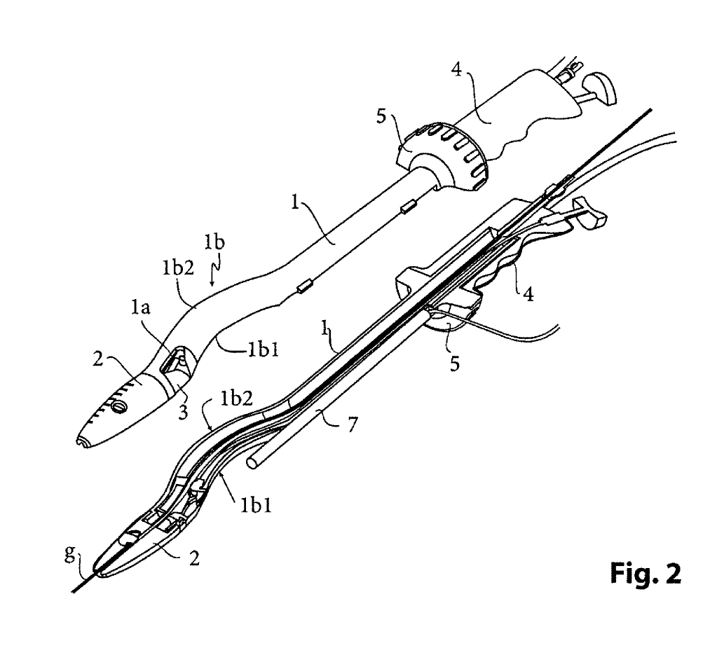

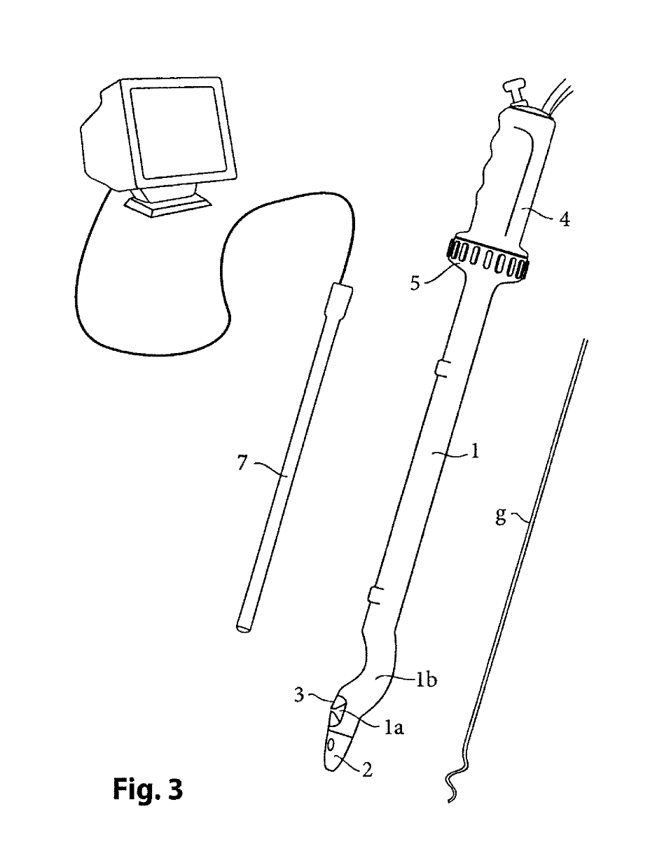

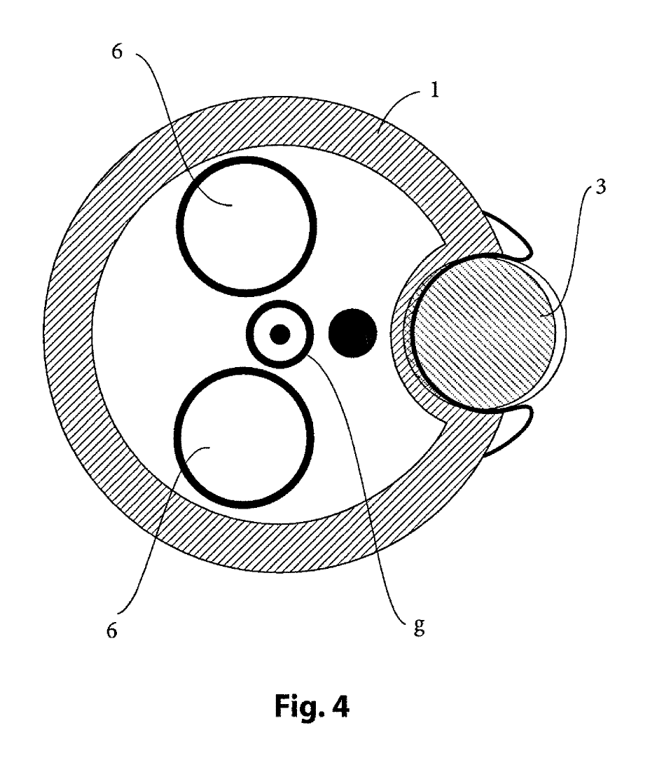

As notably shown in FIGS. 1, 2 and 3, the device comprises a tubular body (1) suitable for being inserted by means of a minimally invasive or endoscopic channel in relation to a previously inserted guide wire (g) in order to pass through the flaps of the native calcified valve above the portion where the calcification is to be removed. Note that the body (1) can be made of a malleable material in order to allow the operator to conform it to the anatomy of the patient. This body (1) is fitted, at the end viewed at the flaps of the valve, with a profiled tip (2), of a soft material, in order to pass through the valve and pre-dilate the central hole of the calcified valve to allow for the passage of the body (1) and to facilitate the positioning of the work area without damaging healthy tissue. The second function of this tip is to limit the lateralization of the ablative portion of the device towards the aortic ring. The decalcification of the valve proceeds with the ablation of tissue from the central hole to the periphery, such as to perform, in contact with the aortic ring, deep decalcification but without lesions within the tissue of the aortic ring: the plastic tip used to cross the valve, once the device is positioned in contact with the aortic ring, is already held against the tissues of the left ventricular outflow tract thus preventing any pressure from the ablative portion of the device from being applied to the aortic ring.

This end, which is equipped with the tip (2), has arrangements for the lateral projection of a cutting member (3) of said tissue. As will be further indicated in this description, this body (3) is operated with only one hand using a handle (4) located externally thereto. The body (1) is mounted free to rotate relative to the handle (4) such that it is capable of being driven manually by means of a toothed wheel (5). The arrangements for the lateral projection of the cutting member (3), consist of an opening (1a), formed at the end of the body (1), at the tip (2).

According to another feature of the invention, the end of the body (1) has, prior to the opening (1a) for the lateral projection of the cutting member (3), a rounded area (1b) in continuity with the said body (1), but laterally offset in relation to the latter. This rounded area (1b), defines on the same side as the opening (1a), a recessed portion (1b1) and, on the opposite side, a projecting portion (1b2) suitable for bearing upon the flaps during the cutting procedure under the effect of a rotational movement of the body (1) by means of the toothed wheel (5) in order to decalcify the whole valve as a result of a circular motion.

In one embodiment, the tip (2) or the end of the body (1) at said tip, is equipped with an adjustable guide means, suitable for gradually engaging with calcified tissue, during the ablation procedure using the cutting system (3), in combination with a spiral trajectory effect applied to said tip (2) or said body.

Tissue ablation is performed following a spiral trajectory from the center to the periphery of the valve by means of the translation or rotation of the cutting system (3) against the edges of the tissue. Decalcification is performed by applying lateral pressure from the cutting system (3) in combination with the guide means (5) against the calcified tissue.

The guide means, for example, comprises soft surgical tape (8) suitable for being deployed in a circular manner in a position of contact with the calcified tissue. Advantageously, the tape (8) is deployed eccentrically with respect to the tip (2). For example, the tape (8) is placed in combination with a rotary shaft controlled by an operating member accessible externally to the body (1), and with a portion that is attached to the tip (2) from wherein said tape is deployed. More particularly, one end of the tape (8) is attached to the rotary shaft to be thus wound onto the latter and to project through an opening of the tip (2) or the body (1) in order to be attached at its other end to the fixed portion of said tip. This fixed portion may consist of a slot such as to allow said tape to project in an eccentric manner. In this embodiment, the laterally offset rounded area (1b) of the body (1) is no longer necessary.

It follows that the cutting member (3), in combination with the rounded area (1b) allows for the cutting off of tissue by translation and rotation against the edge of the opening, from the center to the periphery of the valve as a result of lateral pressure.

The cutting member (3) can constitute a blade, a punch, a drill, an ultrasonic system, etc. and can successively introduce several active areas of different features in order to produce, first, a coarse cut followed by a fine cut or by increasingly finer cuts.

According to another important feature of the invention, the cutting members (3) at the opening (la) of the body (1) are in communication with suction means (6) for the cut tissues. More particularly, the cutting area is combined with a suction cleaning system that is suitable for removing calcification tissue debris upon the ablation thereof, in order to avoid the latter, in whole or in part, being found within the blood circulatory system. The system is put into operation when the cutting member (3) is actuated and obstructs the opening (1a) in order to create a seal with respect to the exterior thereof. Similarly, the tip (2) has a lateral opening (2a) connected to a suction line (6a). These arrangements prevent the device from having to be removed from the aortic root between each cut before having finished the full decalcification of the valve.

Given that the surgical procedure is performed endoscopically, it is necessary to be able to view the area where the intervention is to take place. For this purpose, the device is equipped with a vision system (7) opening into the recessed portion (1b1) of the rounded area (1b) at the level of cutting member (3). This vision system (7) is of any known and appropriate type consistent with those commonly used in the art of endoscopic surgery.

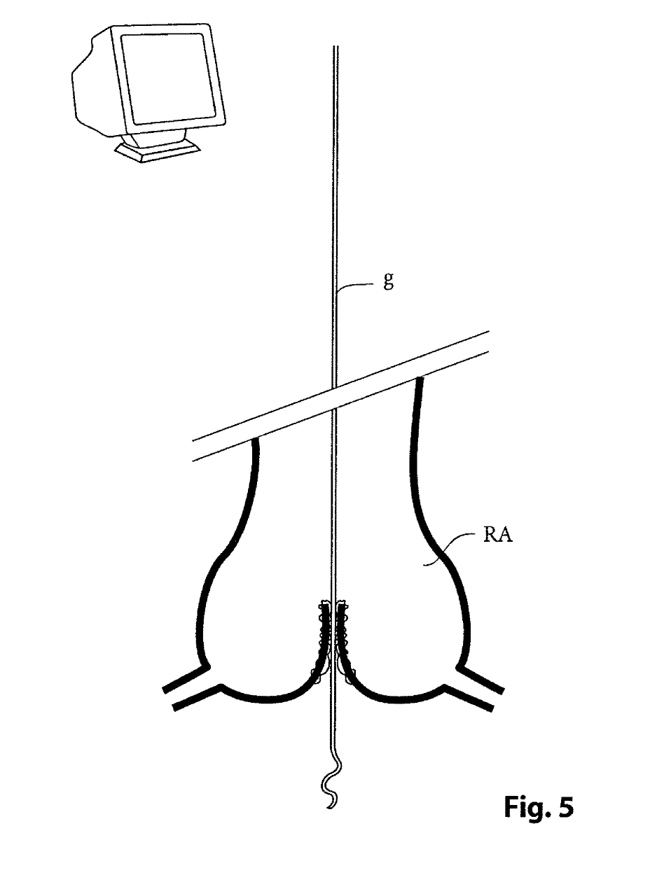

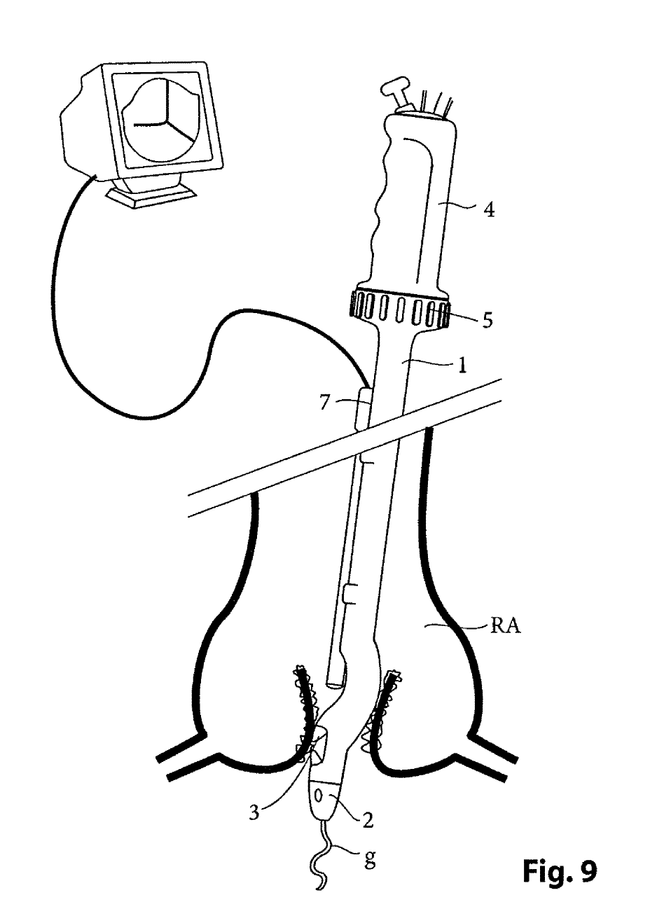

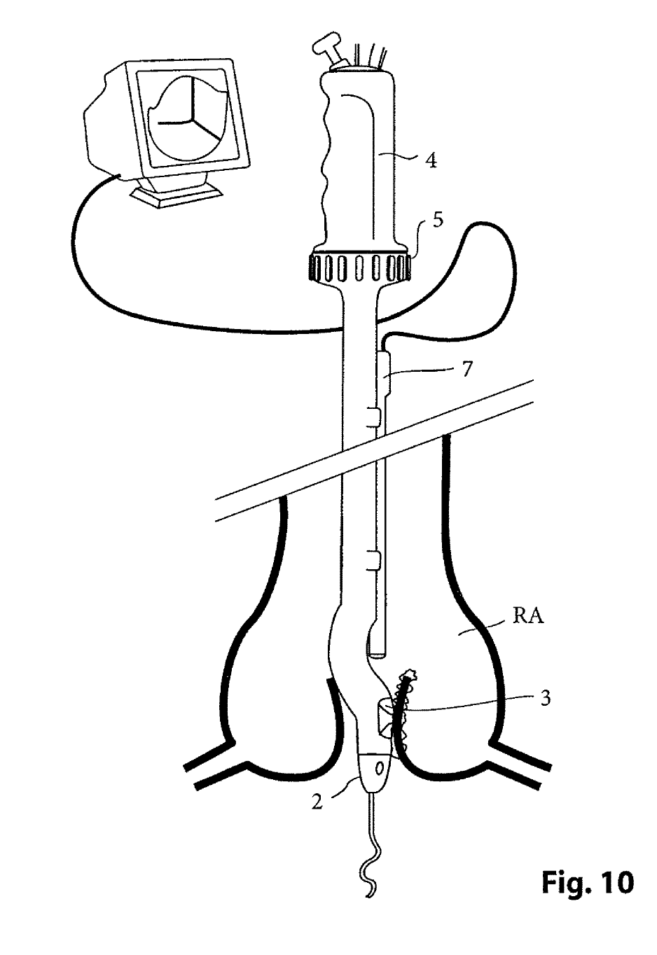

Reference is made to FIGS. 5 to 11 which show the various sequences for the introduction of the device for the ablation of calcified tissue at the level of the valve.

FIG. 5 shows the aortic root (RA) after the positioning of the guide wire (g), which, in a known manner, is engaged such as to cross the stenosed aortic valve.

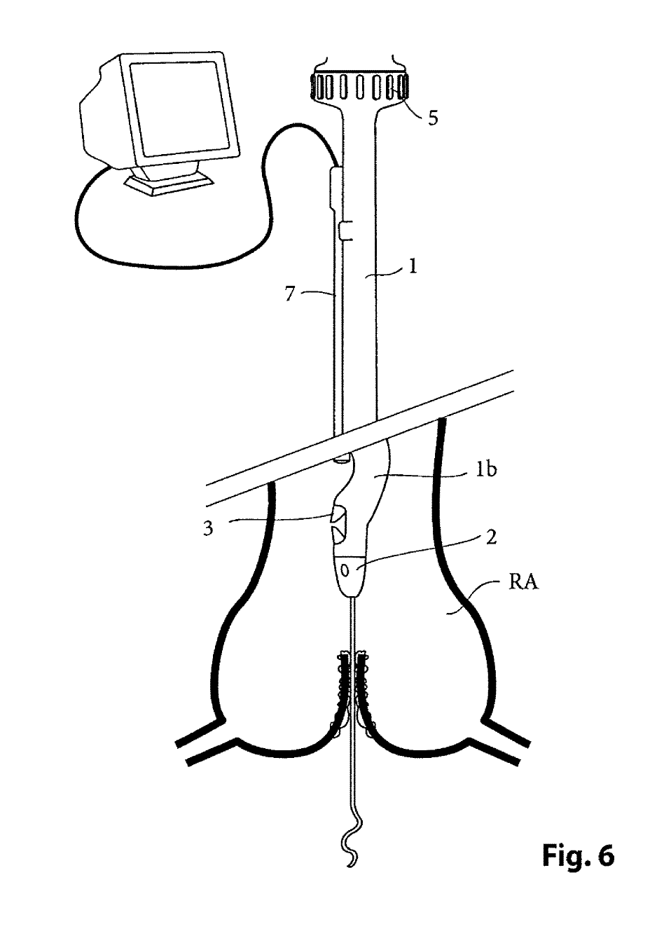

The device equipped with the vision system (7) is then introduced over the guide wire (g) above the portion of the valve where calcification is to be removed (FIG. 6).

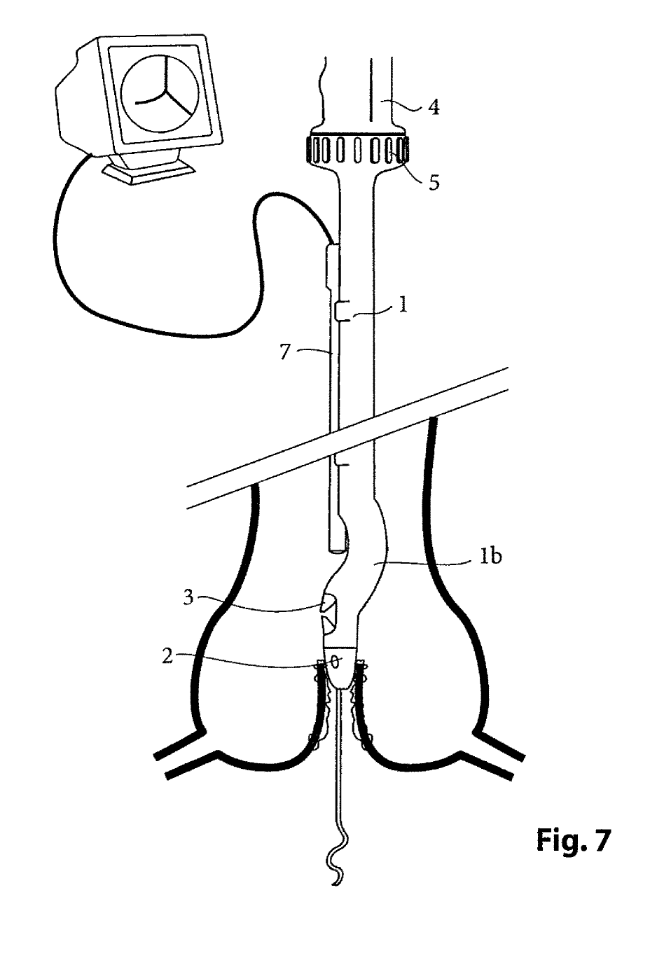

FIG. 7 shows the device in a position wherein the tip (2) is engaged within the calcified area of the flap.

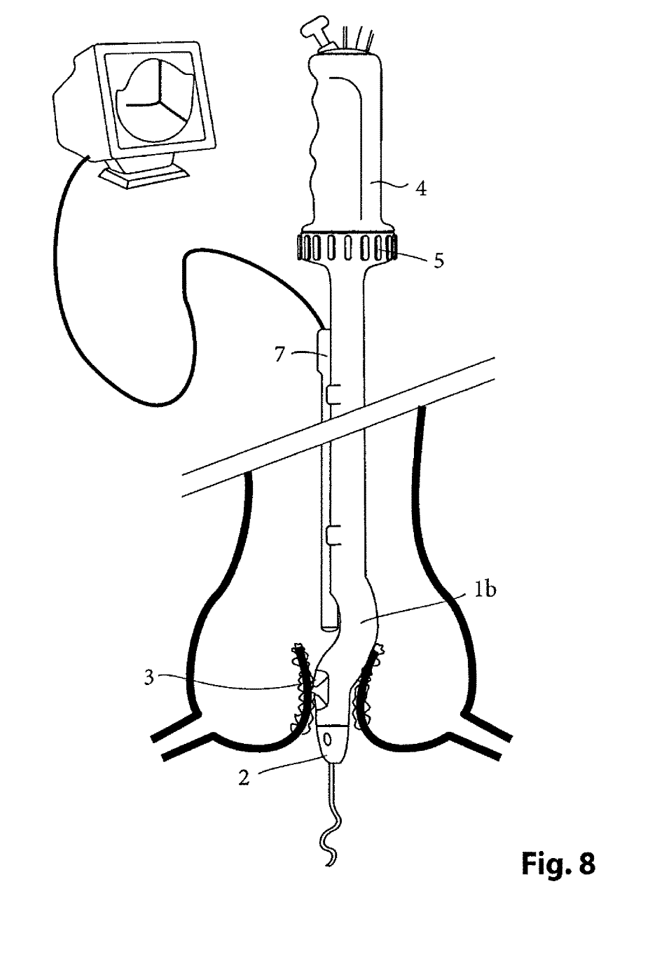

FIGS. 8, 9 and 10 show the ablation procedure itself, of calcified tissue after actuating the cutting member (3) and rotating the body (1) relative to the handle (4), from the center to the periphery of the valve as a result of lateral pressure. As indicated, whenever the cutting device is actuated, the suction cleaning system is put into operation.

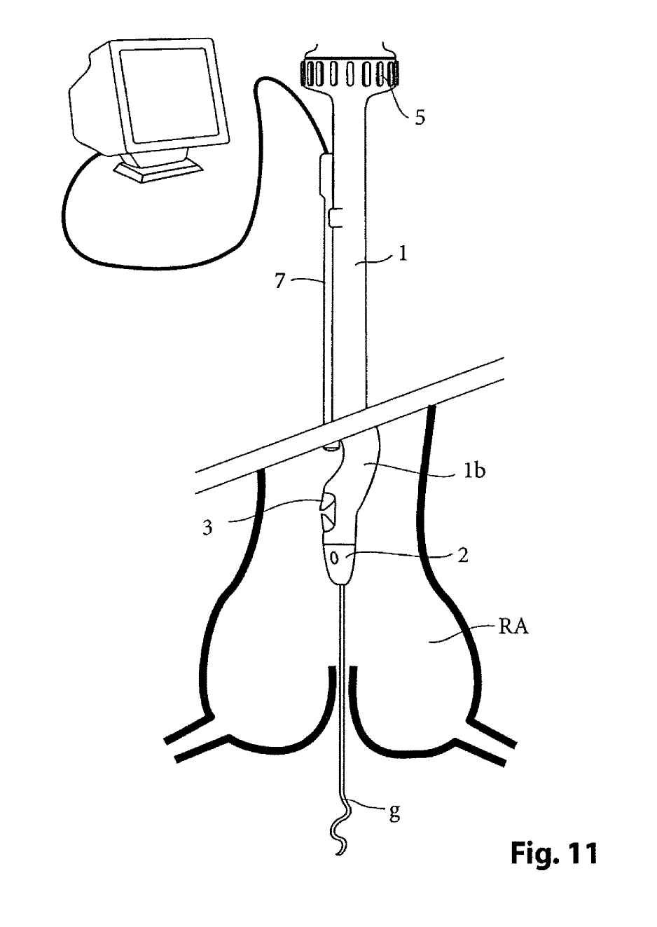

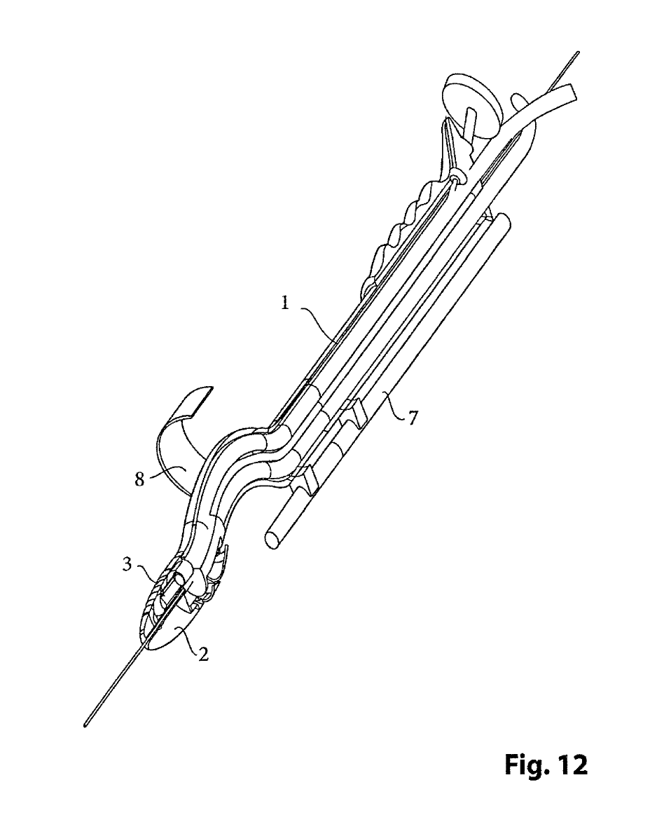

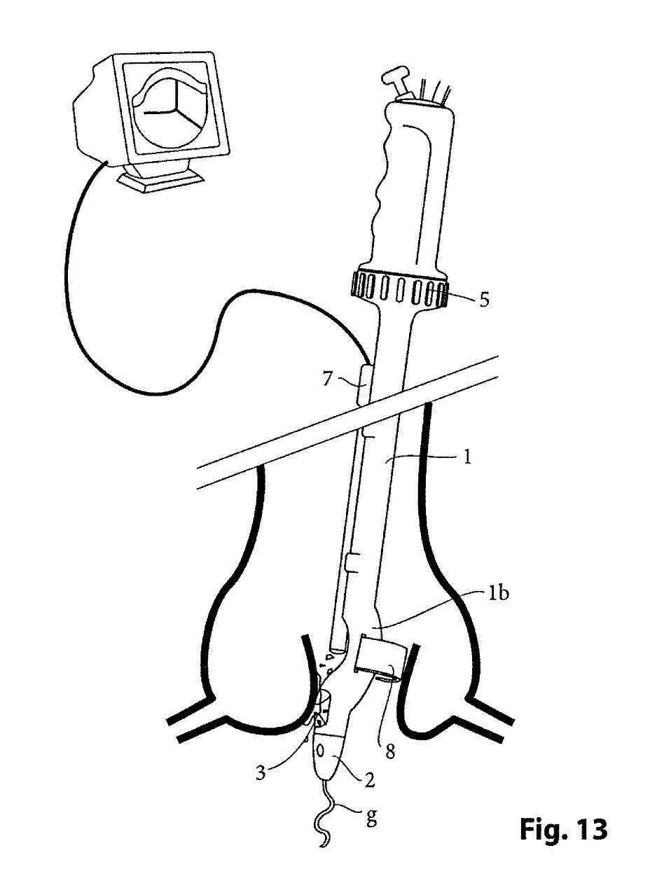

FIG. 11 shows the removal of the device after completing the decalcification procedure. It is then possible to position a prosthetic valve by any known and appropriate means.

The advantages are apparent from the description.

1. A device for cutting off and removing calcified tissue from a heart valve, characterized in that it comprises a tubular body suitable for being inserted by means of a minimally invasive or endoscopic channel in relation to a previously inserted guide wire and suitable for passing through the flaps of the valve above the portion where the calcified tissue is to be removed, one of the ends of said body viewed at said flaps, having a profiled tip made of flexible material and arrangements for the lateral projection of a cutting member for cutting said tissue, the end of the body having, upstream from the arrangements for the lateral projection of the cutting member, a laterally offset rounded area defining, on the same side as the arrangements, a recessed portion and, on the opposite side, a projecting portion suitable for bearing upon the flaps during the cutting procedure under the effect of a rotational movement of the body that can be actuated using only one hand by means of a handle located externally in order to decalcify the whole valve, said arrangements being in communication with means for sucking away the cut tissue.

2. The device according to claim 1, characterized in that the tip or the end of the body at the connection with said tip, is equipped with an adjustable guide means suitable for gradually engaging with calcified tissue during the ablation procedure using the cutting system in combination with a spiral trajectory effect applied to the tip.

3. The device according to claim 2, characterized in that the adjustable guide means is soft surgical tape suitable for being deployed in a circular and eccentric manner in a position of contact with the calcified tissue.

4. The device according to claim 1, characterized in that the body is mounted free to rotate relative to the handle such that it is capable of being driven manually by means of a toothed wheel.

5. The device according to claim 1, characterized in that it has a vision system at the level of the cutting member.

6. The device according to claim 1, characterized in that the body has a means of maintaining the vision system in a stable position during the cutting procedure.

7. The device according to claim 1, characterized in that the arrangements for the lateral projection of the cutting member comprises an opening formed at the end of the body, at the tip.

8. The device according to claim 1, characterized in that the cutting member conforms to the cutting of tissue by means of translation and/or rotation against the edge of the opening, from the center to the periphery of the valve, as a result of lateral pressure.

9. The device according to claim 8, characterized in that the cutting member has successive active areas with different features, in order to achieve, first a coarse cut, then one or more fine cuts.

10. The device according to claim 1, characterized in that it is combined with a washing/suction system that is suitable for removing tissue and calcification debris upon the ablation thereof, said system being put into operation when the cutting member is actuated, and obstructing the opening in order to create a seal with respect to the exterior thereof.

11. The device according to claim 10, characterized in that the tip has a lateral opening that is in communication with a suction line.

12. The device according to claim 1, characterized in that the soft tip is made using a different color to that of the decalcifying area and has a graduation for the precise positioning of the working area with the cutting member.
